(19) United States
(12) Patent Application Publication
(10) Pub. No.: US 2018/0169420 A1
(43) Pub. Date: Jun. 21, 2018

(54) HERMETIC HOUSING AND ELECTRONICS PACKAGE FOR AN IMPLANT DEVICE

(71) Applicant: PIXIUM VISION SA, Paris (FR)

(72) Inventors: Robin REYNAUD, Paris (FR); Eric LE JOLIFF, Paris (FR)

(21) Appl. No.: 15/737,693

(22) PCT Filed: Jun. 17, 2016
(86) PCT No.: PCT/EP2016/001024

§ 371 Date: Dec. 18, 2017

(30) Foreign Application Priority Data
Jun. 19, 2015 (EP) 15001827.3

(51) Int. Cl. A61N 1/375; A61N 1/05

(52) U.S. Cl. CPC A61N 1/3758; A61N 1/36046; A61N 1/0543

(57) ABSTRACT

The present invention relates to a hermetic package (40) suitable to be implanted in a body of an animal or a human patient. The housing (40) comprises a base part (50), a cover part (60) suitable to cover the base part (50), and a connecting means (70), provided at an interface between the base part (50) and the cover part (60). The base part (50) comprises a first hermetic material and the cover part comprises a second hermetic material and the connecting means (70) comprise a third hermetic material, adapted to hermetically seal the interior of the hermetic housing (40) from the outside of the hermetic housing (40). The present invention further refers to an implantable electronics package with such a housing, an implant, in particular a retinal implant, and a method to provide a hermetic housing for an implant.

The invention relates to a hermetic housing and an electronics package for an implant, in particular a retina implant and a prosthesis system, in particular a visual prosthesis system at least partly located in the interior of a patient's eye. The invention further relates to a method for producing such a housing and electronics package for an implant, in particular a retina implant.

There exist a variety of different diseases of the retina that are caused by a degeneration of the photosensitive cells of the retina. Examples of degenerative diseases are retinitis pigmentosa, macula degeneration or Usher syndrome. As a result of these degenerative diseases, people slowly lose their vision and eventually suffer from complete blindness. A visual prosthesis system comprising a retina implant is a helpful tool for at least partially re-establishing a modest visual perception and a sense of orientation for blind and visually impaired users by exploiting the fact that although parts of the retinal tissue have degenerated most of the retina may remain intact and may still be stimulated directly by light dependent electrical stimuli.

In general, the electrical power required for the retina implant's operation and, possibly, data signals are supplied to the implant via a high frequency electromagnetic field. The electro-magnetic field in the case of a retinal implant may, e.g., be generated by a transmission coil that is integrated into an eyeglass frame, i.e. by an extracorporeal device. The retina implant comprises a receiver coil adapted for receiving the high frequency electromagnetic field, wherein the received high frequency signal supplies the power required for the retina implant's operation.

The implanted retina prosthesis system, typically an extraocular implant which may be situated in the orbit of the eye, is adapted to receive the signal and, in response, may thus be supplied with power and is enabled to generate an electrical pulse or an electrical pulse sequence in order to stimulate electrodes on a further implanted device of the prosthesis system, such as an intraocular implant. That intraocular implant receives stimulation pulses based on the scene content received by the extraocular implant. The intraocular implant, in order to enable for stimulation, is provided in proximity or in contact with living tissue or cells, which shall be stimulated, e.g., neural tissue or neural cells, in particular in an eye.

Systems are known, for instance from EP 2 259 843 B1, according to which an extracorporeal implant device is connected with an intracorporeal, extraocular implant. Further, the intracorporeal, extraocular implant is connected with an intraocular implant. The intraocular implant typically is provided epiretinally or sub-retinally, whereas the extraocular implant typically is attached to the sclera of the eye. In order to provide stimulation pulses to the retina by means of the intraocular implant, the extraocular implant comprises at least an electronic device or a power supply capable of translating the information received from the extracorporeal prosthesis device and capable of generating stimulating pulses or stimulating pulse patterns, and transmit those stimulating pulses or stimulating pulse patterns to the intraocular implant.

The electronic device of the extraocular implant, accordingly, needs to be protected against environmental conditions, such that even long-term implantation does not or at least not severely affect the function of the implant. With that respect, it is desired to prevent environmental liquids from entering the implant, as, otherwise, electronic devices may be damaged or destroyed, e.g., due to corrosion.

So far, it was known to use glass solder, such as lead glass solder, which may provide a connection between housing elements. These lead glass solders could be applied and cured at decent temperatures. That may allow the placement of temperature sensitive electronics within a housing, without damaging those electronics during a curing of the lead glass solder in order to hermetically seal the housing. However, lead-containing glass solder generally are not ideal bio-compatible material, and may not provide a hermetic sealing which allows, in particular, a long-term implantation in a body. Accordingly, humidity may ingress and lead to corrosion and destruction of the hermetic seal and metal components. Further, contamination of the body due to the solder components may occur. It is therefore desired to improve both biocompatibility and reliability of such a housing.

What is exemplified for the case of a retinal prosthesis system applies in the same way for other implants, as well, for instance implants to stimulate other tissues such as muscles, e.g., the heart, neural tissue such as the ear, in particular the inner ear, nerves or nerve fibers and others. The present invention may apply for any such applications of implants, as well.

In existing systems, typically, at least a receiving or transmitting coil is provided, which is situated at a remote position from the electronics package. That is required, on the one hand, in order to ensure reliable data transmission and signal reception. Placing the coil close to those housings known from the prior art may cause interferences in the electrical fields and affect capacitances and field distributions of proximate structures.

On the other hand, coils remote from the electronics package may require a very spacious housing, if they were to be arranged within the same housing as the electronics package. Alternatively, these coils may require a separate hermetic housing or coating, which either requires a further spacious housing or may not sufficiently protect the coil from the environmental conditions.

It is therefore an object of the present invention to provide an improved implantable device, which omits at least one of the problems known from the prior art. In particular, an electronics package is desired with increased hermeticity.

It is further desired to provide an implantable device, which is less space-demanding and which allows reliable signal transmission.

It is a further object of the present invention to provide a method, which allows a reliable hermetic sealing of an electronics package.

The problem is solved according to the invention with a hermetic housing according to independent claim 1, an electronics package according to claim 6 and a prosthesis system according to claim 11. Further, the problem is solved by a method according to claim 16. Advantageous developments are subject to the dependent claims.

According to a first aspect of the present invention, a hermetic housing is provided, which is suitable to be implanted into a body of an animal or a human patient. The housing comprises a base part and a cover part. The cover part is suitable to cover the base part. Further, the housing comprises a connecting means. The connecting means is provided at an interface between the base part and the cover part. The base part comprises a first hermetic material and the cover part comprises a second hermetic material, in order to provide a hermetic seal to the housing. Further, the connecting means of the housing comprises a third hermetic material, which is adapted to provide a hermetic seal, thus hermetically sealing the interior of the hermetic housing from the outside of the hermetic housing. The connecting means may, in particular, be provided at an interface between the base part and the cover part, e.g., on an edge of the base part facing toward the cover part, or vice versa.

It needs to be noted that, according to general knowledge, the term “hermetic” may be understood as a seal that is completely gas tight or impermeable to gas flow. However, those skilled in the art will realize that an ideal seal, i.e. an unlimited, application-independent seal, may not be accomplished, in particular in the context of micro structures such as microelectronic mechanical systems (MEMS). According to the present invention, the term “hermetic” may therefore also be used for a sealing which provides a sufficient air tight seal, which will keep gases, moisture or specific molecules out of a housing which is defined to be “hermetically sealed”, for a predetermined time and for a specific application. With that respect, it is referred to the publication “Glass Frit as a hermetic Joining Layer in Laser Based Joining of Miniature Devices”, Qiang wu, School of Electrical and Electronic Engineering, 2010.

There exist various methods to characterize the quality of a hermetic seal. One hermeticity standard, which all of the hermetic housings according to the present invention have to pass is a helium leak test and/or a subsequent gross leak test. Test conditions may for instance be defined by the standard helium fine leak test (such as MIL-STD-883H Method 1014, Mil-Std 750 method 1071, Mil-Std 202 Method 112), or further appropriate tests known to those skilled in the art, such as Gross leak test. Fine and Gross leak testings are widely used in the microelectronic industry. Test criteria may for instance require that in a leak test a leak rate is less than 10⁻⁶ atm-cm³/sec (air), preferably less than 10⁻⁷ atm-cm³/sec (air), most preferably, in particular for devices with a volume of equal to or less than 0.05 cm³, less than 5*10⁻⁸ atm-cm³/sec (air), or below, in particular below 10⁻⁹ atm-cm³/sec (air).

With that respect, a “hermetic material” in the context of the present application is a material which passes those tests for hermeticity applicable for the desired application as set out above, i.e. a hermetic material is enabled to provide a hermetic barrier. In particular, hermeticity within the scope of the present invention shall refer to a hermeticity which allows a long-term implantation for an implant, an electronics package and/or a hermetic housing accommodating electronic components. With that respect, hermeticity may also ideally be defined as a hermetic barrier with hermetic properties to provide a hermetic seal throughout a life-cycle of a product protected by the hermetic barrier, e.g. by a hermetic housing or coating.

It will further be noted that the high hermeticity standards defined above shall in particular apply to the hermetic housing according to the present invention. It is, however, possible that in addition to the housing, a further hermetic housing or layer may be provided around that hermetic housing according to the present invention. In that case, the same hermeticity standards may apply to that additional hermetic cover or housing. However, that additional hermetic cover may also fulfil lower standards, without departing from the scope of the present invention. That may be relevant, for instance, in embodiments, which require some components, such as a transmitting and/or receiving coil, a photodiode, or other components, to be placed outside the hermetic housing. It should nevertheless be noticed that transmitting and/or receiving coil can further be placed inside the housing. In the hermetic housing in contrast, highly sensitive electronic components, such as an electronics chip and connection pads may be accommodated. Those components outside the hermetic housing may be more resistant to environmental effects and thus may allow lower hermetic standards.

By providing connecting means between the base part and the cover part, a hermetic seal may be reliably established, in particular if any components such as electronic components, need to be placed within the hermetic housing prior to covering the base part with the cover part.

In an embodiment of the present invention, the cover part comprises a material, which is transparent to at least a predetermined wavelength or wavelength range. Transparent in this respect means transparent to light or optically transparent. Light may be in the visible light region (e.g. 300-900 nm wavelength). That may allow to manipulate the connecting means with light through the cover part and thus facilitate the hermetic sealing of the housing. The material of the cover part preferably is a metal-free material, in particular a metal-free glass. Omitting the use of a metal-cover may increase the reliability of the housing in a case that electronic components generating, detecting or receiving electrical fields from outside the housing shall be accommodated within the housing. That may in particular be true for components, which are to receive a signal from an extracorporeal prosthesis device, or for components transmitting signals to further implanted components according to some embodiments of the present invention.

Accordingly, the base part of the housing may comprise a ceramic material, preferably a metal-free ceramic material. The use of a ceramic material may in particular be advantageous, as ceramics, such as for instance alumina, zirconia, or silicon carbide, may already show intrinsic hermetic characteristics and thus facilitate the production of a hermetic housing.

In some embodiments of the present invention, the base part of the housing comprises a plurality of layers. Specifically, these layers may be layers of low temperature co-fired ceramic (LTCC). These layers may, for instance, be referred to be their trading name as “DuPont blue tape” or “DuPont green tape”. In particular, at least some of the LTCC layers may have provided electrical contacts and/or through contacts provided therein. That may allow an external contacting of any electronic components provided within the housing.

In particular embodiments, the base part of the housing comprises ten LTCC layers. In these embodiments, four of the LTCC layers may comprise electrical wirings and contacts. These four layers are subsequently provided and form the bottom of the base part. The remaining six LTCC layers may be provided as a ring, e.g., as a circular ring, and together, when subsequently stacked onto each other, form a circumferential wall or rim of the base part. The width of the ring of each of the wall layers is chosen such that a hermetic seal may be provided, once the individual layers are combined.

In another particular embodiment, the base part of the housing comprises twelve LTCC layers. In these embodiments, five of the LTCC layers may comprise electrical wirings and contacts. These five layers are subsequently provided and form the bottom of the base part. The remaining seven LTCC layers may be provided as a ring, e.g., as a circular ring, and together, when subsequently stacked onto each other, form a circumferential wall or rim of the base part. The width of the ring of each of the wall layers is chosen such that a hermetic seal may be provided, once the individual layers are combined.

The connecting means may be a solder paste, preferably a metal-free solder paste. In particular in embodiments comprising a number of LTCC layers stacked onto each other to form the base part of the housing, that solder paste may be provided on the uppermost LTCC layer, i.e. on a surface of the base part which faces the cover part. The solder paste may also be pre-deposited and pre-cured onto that first LTCC layer.

Providing the connecting means by way of a paste, i.e., a solder paste, may allow facilitated handling of the housing and the defined application of the solder during the process of hermetic sealing. In particular, using a solder paste may allow to print the solder onto the base part and/or onto the cover part, which may facilitate the production of the housing.

It should be noted that the term “metal-free” shall refer to materials, which do not have electrical properties as they are typical for metals, such as electrical conductivity, inductivity or similar. It is to be noted however that materials, such as ceramics, may be used that comprise metal ions, such as alumina, i.e. aluminium oxide. These materials, however, do not have characteristics comparable to those of elemental metal and as such do not interact with electrical fields, or at least do not significantly affect electrical fields. Within the scope of the present invention, therefore, metal-free materials shall be understood as materials that do not or neglectably interfere with electrical fields due to their intrinsic electrical properties as known from elemental metal.

In particular the use of a glass solder, such as for instance glass solder Ferro FX 11-036 or Ferro DL 11-205 may be advantageous to omit corrosion and thus a decrease of hermeticity of the connecting means. Preferably, that glass solder is lead-free, such as Ferro DL 11-205. That may increase the lifetime of the solder paste, the seal provided therewith and, finally, the lifetime of the entire housing including any electronic components accommodated therein. Further, advantageously, the glass solder used is biocompatible. That may allow implantation of the housing. The solder may also be a non-crystallizing solder, in particular solder paste.

However, biocompatible, lead-free solder pastes may have curing temperatures of several hundred degree, e.g. in the range of 250-400° C., in particular between 290-360° C. Such curing temperatures for the solder are beyond acceptable limits granting stability for the bondings provided between electronic components in the housing, such as chips, solder bonded or wire bonded to connection pads. These connections may already begin to deteriorate due to melting at temperatures beyond about 100° C. Since an access to the interior of the housing is not possible after hermetically sealing the housing, the housing together with the electronic components therein may be rendered useless when heating the device to temperatures of more than hundred or even 200° C. Therefore, it is desired to provide an alternative method to cure the glass solder, without overheating the remaining components of the housing.

According to further embodiments of the present invention, the solder paste is light absorbent for a predetermined wavelength or wavelength range. In particular, the solder paste is light-absorbent at least for one wavelength or a part of a wavelength range, for which the cover part of the hermetic housing is transparent. The term “light absorbent” shall in particular also refer to laser light. A laser-beam absorbing solder paste or, generally, a light or energy absorbing solder paste, may thus easily be heated and, eventually, cured, by application of light of a predetermined wavelength. Light, advantageously, is provided by means of a laser in that the laser light is focused on a spot or a region, which shall be heated or cured. Adjusting the light absorption to a wavelength or wavelength range, in which the cover part of the housing transmits the light, a curing of the connecting means, i.e. the solder paste, may be conducted while the cover part is located on top of the solder paste, covering the base part of the housing. A preferred wavelength range may be infrared, in particular near-infrared, light. The laser may for instance be an ILT laser or continuous wave diode laser at a wavelength of 808 nm. Further lasers may be used, as well, without departing from the scope of the present invention.

The laser may in particular be controlled such that it uniformly heats the solder, e.g. by providing a light application with the specific shape of the solder on the base part or by scanning the laser over the solder paste at such a speed that the heating of the solder essentially is uniform. Such laser control may in particular allow equal bonding of the solder to the base part and the cover part. It may further reduce any thermal stress in particular in the cover part, caused by uneven heat distribution within the solder, i.e. the connecting means.

The solder may be applied on an LTCC layer prior to the final assembly of the housing. In particular, the solder may be a solder paste, which requires double curing. Therefore, an

LTCC may be used, having a pre-cured solder paste layer provided thereon. That may help to regulate the thickness of the solder. Advantageously, the solder may be provided with a thickness of typically between about 15-30 μm and 70 μm, preferably between about 30 μm and about 80 μm, more preferably between about 47 μm and about 73 μm after an initial curing of the solder. During curing, the thickness of the solder layer may be reduced, e.g. due to a loss of water in the solder paste and due to the spreading of the solder caused by external pressure and heat. The solder paste layer therefore typically is provided with a thickness, such that, after the pre-curing step, the thickness of the solder on the LTCC layer is about 70 μm, preferably about 60 μm.

According to a second aspect of the present invention, an electronics package for an implant device is provided. The electronics package comprises at least one receiving unit, an electrical circuit adapted to generate a stimulating signal, and a first hermetic housing. The hermetic housing may in particular be a hermetic housing according to the first aspect of the present invention. The first hermetic housing comprises a base part adapted to receive the electrical circuit and/or the at least one receiving unit. Further, the hermetic housing comprises a cover part adapted to cover the base part. The cover part may, e.g., be a lid or a frit, in particular a glass lid or glass frit. In addition, a connecting means is provided between the base part and the cover part. The connecting means is adapted to connect the base part and the cover part and is adapted to hermetically seal the interior of the hermetic housing from the exterior of the hermetic housing.

The implant may be an implant for electrical stimulation of living tissue or cells. In particular the implant may be a retinal implant which at least comprises a first, extraocular implant device and a second, intraocular implant device. The implant may be part of a prosthesis system, which in addition to a first and second implantable device, may comprise an extracorporeal prosthesis device. It should be noted that although, preferably the first implant is an extraocular implant, that first implant may be provided within an eye of a patient, e.g., out of the optical pathway of the eye.

An electronics package for an implant with such a hermetic housing may comprise increased hermeticity (meaning hermetic tightness). Electronic components may therefore be better protected from environmental effects. That may allow to increase the lifetime of the electronics package. Providing a hermetic seal may also reduce deterioration of the seal itself, which may increase its lifetime and, thus, the lifetime of the entire package, as well.

In a development of the present invention, the electronics package comprises a second hermetic housing or cover arranged, at least partially, around the first hermetic housing. By providing a second hermetic housing, for one, the protection from the environment of any component within the first hermetic housing may further be increased. In addition, components, which do not require the same level of hermetic protection as those components, which need to be provided within the first hermetic housing, may be placed outside that first hermetic housing. That way, the size required for the first hermetic housing may be reduced. The hermeticity standards for that second housing may also require the passing of a hermeticity test. The hermeticity test for a hermetic coating or cover for components disposed outside the hermetic housing, such as an intraocular implant with stimulating electrodes and/or a photodiode, may require lower, equal or higher standards than the hermeticity tests required for the first, inner hermetic housing. Typically, such coatings provide a lower hermeticity and standards and tests are of different scope.

According to some embodiments, outside the first hermetic housing and inside the second hermetic housing at least one transmitting and/or receiving unit is provided. As previously indicated, different components may require different levels of protection, i.e., protection by means of a hermetic housing or cover. As a matter of fact, providing different electrical components, such as, e.g., different transmitting and/or receiving units close to one another, may lead to interference and compromised signal transmission. Separating transmitting and receiving units locally may help to improve signal transmission and thus may enhance reliability of the electronic components. For those reasons, it may be desirable to place one or more of these units outside that first hermetic housing, to be protected by the second hermetic housing or cover. It will be noted that the sending and/or transmitting units may, in particular, be provided as coils.

In some embodiments, the second hermetic housing comprises a biocompatible material, in particular silicone. Such choice of material may allow long-term implantation of an electronics package within a body, without immune response from the body. Further or alternative materials may be used in addition or alternatively for the second hermetic housing.

According to some embodiments of the present invention, the base part comprises a bottom part, wherein that bottom part comprises a stack of layers. Preferably, at least one of these layers comprises an integrated electrical circuit. By means of the layer structure for the bottom of the hermetic housing of the electronics package, an electrical connection may be established to the outside of the housing. At the same time, the stack of layers may provide hermetic sealing of the housing. That way, at least the most sensitive electronical components may be securely protected within the housing of the electronics package, while an electrical connection to external components is enabled.

According to a third aspect of the present invention, a prosthesis system, in particular a prosthesis system for a retinal prosthesis, is provided. The prosthesis system comprises as a first implantable device an electronics package according to the second aspect of the present invention. The first implantable device is adapted to be implanted into a body of a patient. The prosthesis system further comprises a second implantable device, which is adapted to be implanted into an organ, preferably an eye, of a patient, and being connected with the electronics package. Accordingly, the prosthesis system comprises at least two components, a first component comprising an electronics package and a second component adapted to be implanted directly in an organ. Those skilled in the art will understand that the first component of the prosthesis system may be situated, when implanted, inside a body, but outside an organ which shall be supported in its functionality. In the example of a visual prosthesis system, the first component may, therefore, be an extraocular, yet implanted device.

The second implantable device may comprise at least one stimulating electrode capable of stimulating living tissue or cells. That may allow to directly stimulate specific tissue regions or even individual cells of a designated tissue. Further, the first or, preferably, the second implantable device may comprise a photodiode, which is adapted to receive and detect light transmitted from e.g. outside of an eye onto the photodiode. That way, information may be submitted to the implant.

Notably, by providing a first implant component and a second implant component, the second implant component may be reduced in size significantly, as all necessary electronic components e.g. for stimulating the tissue, may be arranged in the first implantable device. Thus, the second implantable device, which is intended to be located within or in close proximity to the tissue in question, may be implanted less invasively. Further, by providing a separate first implantable device, space-consuming electrical components, such as energy supply, signal generator and others, may be placed e.g. outside of the organ or at a remote position within the organ, where a negative effect on the patient and on the function or residual function of the organ or the tissue within the organ is reduced.

In an embodiment of the present invention, the second implantable device comprises a receiving unit, which is connected to a transmitting unit, of the electronics package. Accordingly, the connection of the transmitting unit of the electronics package, i.e. the first implantable device, with the receiving unit of the second implantable device may be a wire-connection or a wireless connection. In particular in the case of a wireless connection between the first and the second implantable device, the transmitting unit and the receiving unit may be a transmitting coil and a receiving coil, respectively, or they could be both a sending and receiving coil. It will be noted that both, the first and the second implantable device may have a sending and receiving unit, such that a two-ways communication between the first and the second implantable device may be established. That way, the second implantable device may also be used as a sensor.

According to further embodiments of the present invention, the prosthesis system comprises an extracorporeal component, which comprises at least a first transmitting unit and a signal generation unit. The signal generation unit is adapted for generating a signal and applying the signal to the at least first transmitting unit of the extracorporeal component. The at least one first transmitting unit is adapted for transmitting the signal generated by the signal generation unit to the electronics package.

Within the context of the present invention, the term “extracorporeal” shall be understood to define components according to the present invention, which, when used e.g. as a part of a prosthesis device, are intended to be placed outside of a body of a patient or an animal. Thus, according to the present invention, it may be differentiated between three categories of devices of a prosthesis system, namely extracorporeal components, first implantable devices, i.e. components implanted into a body, preferably outside of specific organs, and second implantable devices, i.e., implants, which are implanted within an organ such as an eye. Specifically, in the preferred case of a retinal implant, an extracorporeal device may be a device integrated in a frame of spectacles, or may be attached to the outer epidermal layer of the body. A first implantable device, e.g., an electronics package, may be adapted to be situated within the eye socket, but outside of the eye, i.e. outside of the vitreous body of the eye. For instance, the first implantable device may be adapted to be attached to the sclera of the eye. The second implantable device may be an intraocular device, which is located within the eye, e.g., within the vitreous body of the eye, preferably on, in or under the retina of the eye.

Accordingly, a retinal prosthesis system according to the present invention may comprise three components, an extracorporeal component, an extraocular implant device and an intraocular implant device. All of these components may be connected by means of a wireless connection, a wire connection, or a combination thereof. In specific embodiments and depending on the specific application, the prosthesis system may also comprise only two of these components as indicated above, while at least one of these components comprises an electronics package as set out above, without departing from the scope of the present invention.

According to some embodiments, the first implantable device, i.e., the extraocular implant device, comprises at least a receiving coil, which is adapted to receive a high frequency signal and/or a superposed signal comprising two or more high-frequency signals.

The receiving coil of at least one of the implantable devices may further be adapted to receive electrical energy by means of a signal transmitted from the external, i.e. extracorporeal, prosthesis device. Likewise, in case two implantable devices are used, one of the implantable devices, e.g. the second implantable device such as an intraocular implant, may comprise a receiving unit adapted to receive electrical energy and/or data signals transmitted from the first implantable device, e.g. an extraocular implant.

The electrical circuit of the electronics package may in particular comprise a stimulation chip, which is powered by an internal power supply or directly by means of electrical energy received by the receiver unit.

A fourth aspect of the present invention refers to a method for providing an implantable electronics package. The method comprises the step of providing a base part of a hermetic housing adapted to receive an electrical circuit and/or at least one receiving unit. The base part may, in particular, be a base part of a hermetic housing. The receiving unit may comprise at least one transmitting and/or receiving coil. The electrical circuit and/or the at least one receiving unit is provided on a bottom part of the base part, i.e. within the hermetic housing. Further, on a surface of the base part, a connecting means is provided. Notably, the connecting means may already be provided on the base part prior to the electrical circuit and/or the at least one receiving unit being provided in the base part. In particular, the connecting means may be provided on the base part already during manufacture of the base part.

In an assembly step, a cover part is provided on the connecting means. The cover part is connected with the base part by means of the connecting means, that way hermetically sealing the gap between the cover part and the base part. That connection is established by light induced heating of the connecting means.

In an embodiment of the present invention, the bottom part of the base part may comprise an integrated circuit, electrical connections and/or wire traces for interconnections. In particular, the bottom of the base part of the housing or the entire base part of the housing may comprise a plurality of layers, which, at least on the bottom, may have through-connections to provide an electrical connection from within the hermetic housing to the outside. In such an embodiment, a further step of the suggested method is to connect the electrical circuit and/or the at least one receiving unit, placed within the housing, with the bottom part of the base part.

The electrical circuit may be any electrical circuit or micro-circuit, including an electronics chip, flip-chip or further interconnected electrical components. In particular, the electrical circuit may be a stimulation chip for providing stimulation impulses or data to stimulation electrodes, e.g. to an electrode array.

According to some embodiments of the present invention, the connecting means is a solder paste. In such embodiments, the step to connect the cover part with the base part may comprise laser-soldering of the solder paste. Laser-controlled soldering may provide various advantages. For one, laser light may be focused on very small areas and structures, with high light-intensities, which allows a local heating. Further, the control of a laser may be more precise than for other light sources, in particular in terms of intensity control, response time, or displacement control. That way, only predetermined areas of the housing may be heated, i.e. the areas comprising the solder paste. More remote regions, such as the interior of the housing, are not heated, or are at least not directly heated. That may increase the lifecycle of the electrical components within the housing, which may be damaged in common heating procedures. The method according to the present invention may therefore allow a pre-positioning, contacting and wiring of the electrical components within the housing as compared to methods requiring heat application to the entire housing. That may facilitate the production process.

The solder paste may be pre-cured onto the base part of the hermetic housing. That may facilitate the terminal assembly of the hermetic housing. Further, a solder paste may be applied, which requires multiple curing, such that the curing process in the fully assembled housing may be reduced to a minimum, thereby reducing the risk of damaging the electronic components during heating or provision of a solder paste layer.

The light, in particular the laser, may be controlled to heat only the interface area between the cover part and the base part covered with the connecting means. The light intensity preferably is increased and decreased linearly at least around a desired target intensity at a predetermined target point or target area at the interface between the cover part and the base part.

Advantageously, a solder s used that allows printing of the solder to the base part and/or the cover part by known methods, such as from printed circuits or similar.

Further details, preferred embodiments and advantages of the present invention will be found in the following description with reference to the drawings, in which:

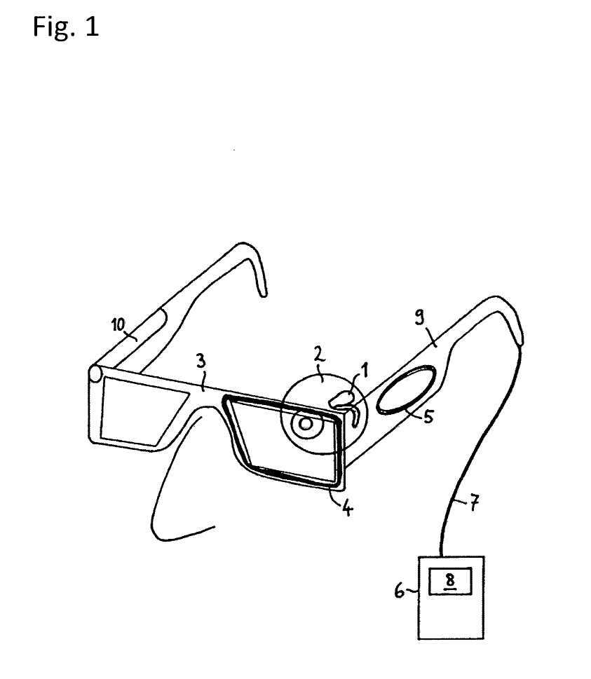

FIG. 1 gives an overview of a visual prosthesis system;

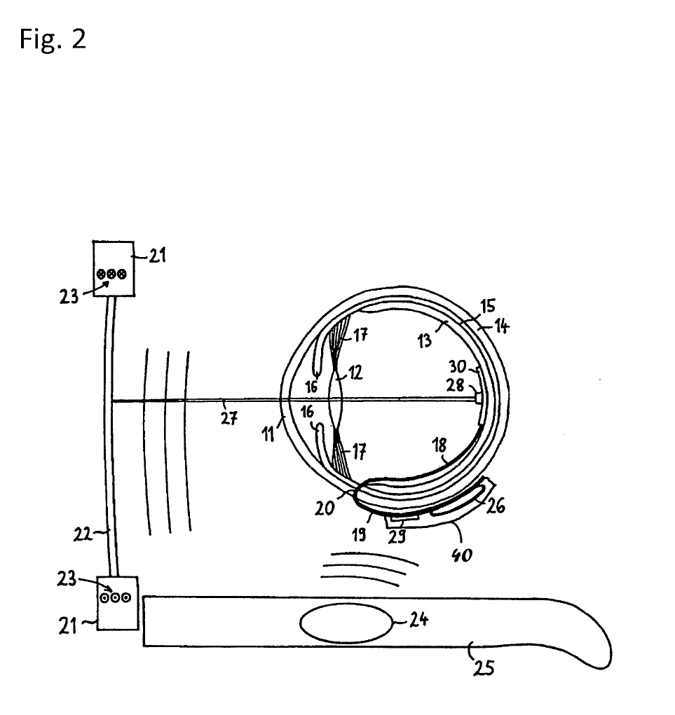

FIG. 2 shows a cross section of an eyeball comprising a retina implant;

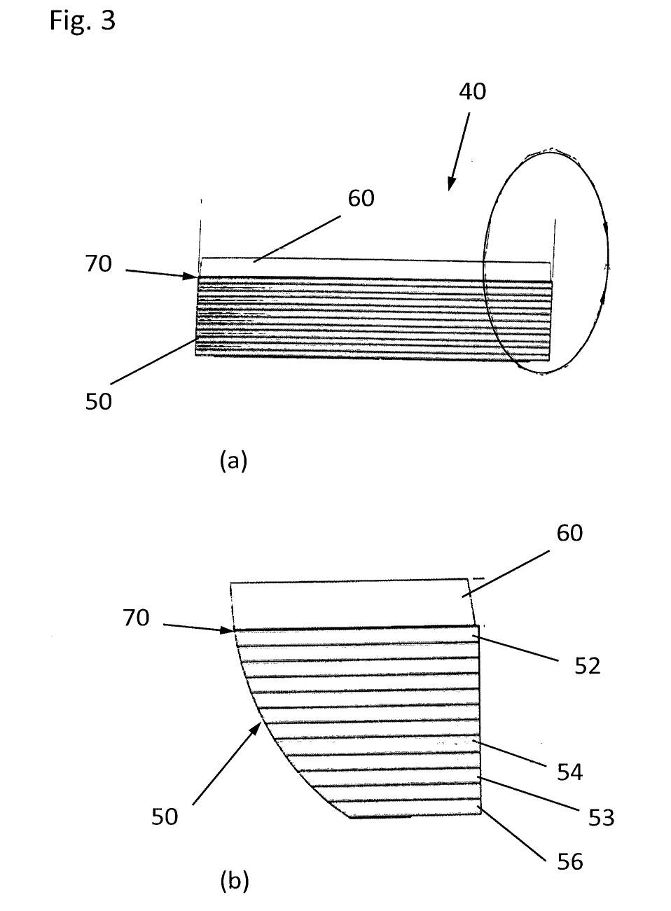

FIG. 3 shows (a) a side view of a hermetic housing according to an embodiment of the present invention and (b) a magnification of a portion of the side view according to (a);

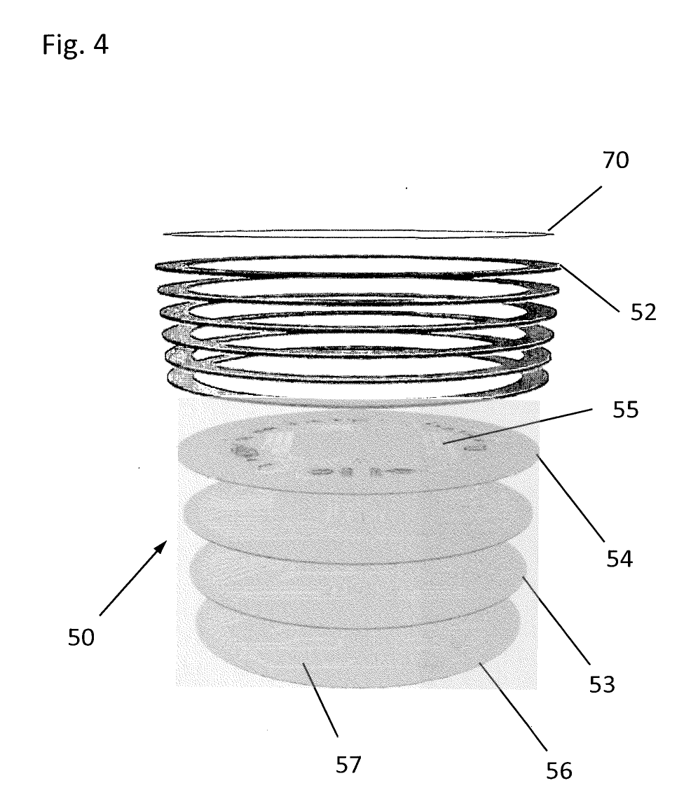

FIG. 4 shows a base part of a hermetic housing according to an embodiment of the present invention comprising a multiple layer structure in an explosive view;

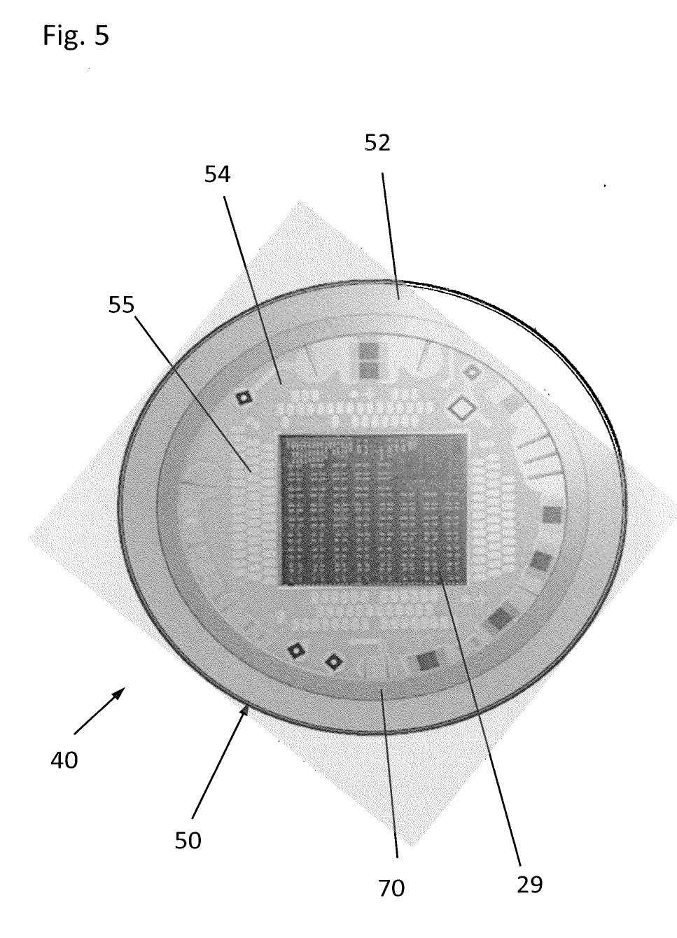

FIG. 5 shows a top view of a hermetic housing according to an embodiment of the present invention.

FIG. 1 shows as an Example a visual prosthesis system for at least partially reestablishing a modest visual perception and a sense of orientation for blind and visually impaired users. There exist a variety of different diseases of the retina that are caused by a degeneration of the photosensitive cells of the retina. Examples for degenerative diseases are retinitis pigmentosa, macula degeneration or Usher syndrome. As a result of these regenerative diseases, people slowly lose their vision and eventually suffer from complete blindness.

The visual prosthesis system shown in FIG. 1 comprises a retinal implant 1 that may for example comprise an intraocular part located within the eyeball 2 and an extraocular part located at the outer surface of the eyeball 2. The intraocular part of the retinal implant 1 comprises an array of micro-contacts that is in direct contact with the patient's retina, wherein the micro-contacts are adapted for electrically contacting the retinal tissue.

The visual prosthesis system further comprises a visual interface 3, which may for example be realized as an eyeglass frame. The visual interface 3 is adapted for supplying energy to the retina implant 1, and for performing wireless data communication with the retina implant 1.

The energy transfer from the visual interface 3 to the retina implant 1 is effected by a first transmission coil 4 and a second transmission coil 5 which are both integrated in the eyeglass frame 21, e.g, a temple arm 9. The visual prosthesis system as shown according to the embodiment of FIG. 1 comprises a pocket computer 6 that is connected to the visual interface 3 via a wire connection 7. The pocket computer 6 comprises a signal generation unit 8 that generates a first high frequency signal for the transmission coil 4 and a second high frequency signal for the second transmission coil 5. Preferably, the two high frequency signals have the same frequency, with the frequency of the first and the second high frequency signal being in the range between 100 kHz and 100 MHz. Further preferably, the second high frequency signal is phase shifted relative to the first high frequency signal. In alternative embodiments, only one transmission coil and, hence, one high frequency signal may be provided.

Via the wire connection 7, the first high frequency signal is supplied to the first transmission coil 4, and the second high frequency signal is supplied to the second transmission coil 5.

The first transmission coil 4 transmits the first high frequency signal, and the second transmission coil 5 transmits the second high frequency signal. The first and the second transmission coil 4, 5 radiate an electromagnetic field having a frequency in the radio frequency range.

The retina implant 1 comprises a receiver coil for receiving the electromagnetic field generated by either the first transmission coil 4 or the second transmission coil 5, or both. The electromagnetic signal received by the receiver coil provides the electrical power for operation of the retina implant 1.

The visual interface 3 may further comprise a video camera 10 for acquiring a video image of the patient's field of view. Video signals acquired by the video camera 10 are transmitted to the pocket computer 6. There, the video signals are converted into corresponding stimulation data for the array of micro-contacts on the retina implant 1. The stimulation data determined by the pocket computer 6 is forwarded to the visual interface 3 and transmitted to the retina implant 1. Alternatively, integrated circuits may be provided, which are enabled to convert the received video signals into stimulating pulses. Accordingly, the pocket computer may also be replaced by a computer or computer chip integrated in at least one of the prosthesis devices, implantable or external to a body. Further, the video signal may be transmitted to a remote computer or computing device, including, for instance a cell phone or a standalone unit. The transmission may in particular be wireless, in order to omit any wire connection affecting a wearing comfort.

For transmitting the stimulation data to the retina implant 1, there exist different alternatives. According to one embodiment, the stimulation data is modulated onto at least one of the first and/or the second high frequency signal. At the retina implant, the received electromagnetic signal is demodulated. In this embodiment, the first and/or the second high frequency signal are used both for data communication and for transferring energy to the retina implant 1.

According to another embodiment, the stimulation data is transmitted to the retina implant 1 via a modulated light beam, preferably via modulated infrared light. In this embodiment, the first and/or the second high frequency signals are solely used for transferring energy to the retina implant 1.

At the retinal implant 1, the stimulation data is decoded. In accordance with the stimulation data, stimulation pulses are applied to the micro-contacts of the retina implant 1. The stimulation of the retinal tissue causes a visual impression.

FIG. 2 shows a cross section of a patient's eye comprising a retinal implant. External light passes the cornea 11 and the eye lens 12 and strikes the retina 13. The retina 13 covers a large part of the eyeball's interior. The eyeball's outer surface is formed by the sclera 14. Between the retina 13 and the sclera 14, a choroid membrane 15 is located. The iris 16 determines the amount of light that may enter into the interior of the eye. The eye lens 12 is fixed by the ciliary muscle 17.

The retina implant according to the embodiment shown in FIG. 2 comprises an intraocular part 18 and an extraocular part 19. The intraocular part 18 is located in the interior of the eye, whereas the extraocular part 19 is fixed to the outer surface of the sclera 14. In the embodiment shown in FIG. 2, the intraocular part 18 and the extraocular part 19 are electrically connected by wire connections 20 that pass through the sclera 14 at a position right behind the ciliary muscle 17. Alternatively, the intraocular part 18 and the extraocular part 19 may be connected wirelessly.

The patient wears an eyeglass frame 21 with glasses 22. A first transmission coil 23 is arranged around one of the eyeglasses. A second transmission coil 24 is integrated in one of the temples 25 of the eyeglass frame 21. That way, the transmission coils have an angular arrangement with respect to another. The first transmission coil 23 is adapted for transmitting a first high frequency signal, and the second transmission coil 24 is adapted for transmitting a second high frequency signal. The electromagnetic field generated by the first transmission coil 23 is superposed with the electromagnetic field generated by the second transmission coil 24. The extraocular part 19 of the retina implant comprises a receiver unit, here a receiver coil 26. The receiver coil 26 is adapted for receiving the superposed electromagnetic signal and for supplying electrical power to the components of the retina implant. Energy transfer from the first and/or the second transmission coil 23, 24 to the receiver coil 26 can be optimized by adjusting the relative phases and the respective amplitudes of the first and the second high frequency signal. Thus, the superposed electromagnetic field can be adjusted to the orientation of the receiver coil 26 in some embodiments of the present invention.

Additionally, stimulation data carrying visual information has to be transmitted from the visual interface to the retina implant. In the embodiment depicted in FIG. 2, a modulated infrared beam 27 is used for transmitting the stimulation data to the retina implant. The infrared beam 27 may for example be generated by an infrared transmitter LED located in the vicinity of the glasses 22. The modulated infrared beam 27 passes through the eye lens 12 and strikes an optical receiver element 28 (e.g. a photodiode) located on the intraocular part 18 of the retina implant. The stimulation data received by the optical receiver element 28 is forwarded via the wire connection 20 to a retina stimulation chip 29 located on the extraocular part 19 of the retina implant, i.e. in a hermetic housing 40 of the retinal implant. Preferably, the retina stimulation chip 29 is implemented as a digital signal processing chip. The retina stimulation chip 29 is operative to convert the stimulation data into corresponding stimulation pulses for an array 30 of micro-contacts located directly on the retina 13. The stimulation pulses are supplied to the array 30 of micro-contacts via the wire connection 20. The micro-contacts are adapted for stimulating the ganglia of the retina 13, and this stimulation causes a visual impression.

According to an alternative embodiment, instead of transmitting the stimulation data to the retina implant via a modulated infrared beam 27, the stimulation data may be modulated onto at least one of the first and the second high frequency signal. According to this embodiment, the first and the second high frequency signal are adapted both for transferring energy and for transmitting the stimulation data to the retina implant.

The receiver coil 26 and the stimulation chip 29 arranged extraocular as shown in FIG. 2 are provided in a hermetic housing 40, in order to reduce any degenerative effects on the electronics. According to the definition of the term hermetic given above, the hermetic housing 40 fulfills at least the standards of hermeticity as set out above, e.g. the MIL-STD-883H Method 1014-standard.

It will also be noted that according to alternative embodiments, an additional transmitting and/or receiving unit, in particular at least one coil, may be provided within the hermetic housing 40. Further, additional or alternative electronic components may be provided within the hermetic housing 40 without departing from the scope of the invention to provide a hermetic housing for an implant.

FIG. 3 shows in its sub-figure (a) a side view of the hermetic housing 40 according to an embodiment of the present invention. The housing 40 comprises a base part 50, a cover part 60, such as a lid or a frit, and a connecting means 70. The connecting means 70 is provided between the base part 50 and the cover part 60 and is suitable to hermetically seal the gap between the cover part 60 and the base part 50.

The sub-figure (b) of FIG. 3 shows a magnified view of an edge portion of the housing 40 as indicated by the ellipse in FIG. 3(a). The base part 50 of the housing 40 in the embodiment according to FIG. 3 comprises a layer structure. The layer structure therein comprises at least an outer bottom layer 56. The outer bottom layer 56 is the outermost layer, which may therefore enable a contact to the outside of the hermetic housing, as will be discussed with respect to FIGS. 4 and 5. On top of the outer bottom layer, multiple intermediate layers 53 may be provided. As an innermost layer defining a bottom of a cavity of the housing 40, an inner bottom layer 54 is provided.

On top of the inner bottom layer 54, in the embodiment of FIG. 3, a plurality of wall layers is provided, comprising a top wall layer 52. As may be seen in FIG. 4, the wall layers are ring-like shaped, forming a cylindrical cavity on the inner bottom layer, when stacked onto another. Electrical components to be protected by the hermetic housing may be placed within that cavity.

On top of the top wall layer 52, the connecting means 70 is provided as an additional layer. The cover part is placed on the top wall layer 52, thus sandwiching the connecting means between the cover part 60 and the base part 50.

As previously indicated, the cover part may comprise glass. In particular, the cover part may comprise a glass which is transparent to light, e.g. infrared light of the near infrared range. The cover part accordingly may be transparent to more than 90% of incident light of a near-infrared wavelength, e.g. between 800 and 940 nm. Further, in order to limit the size of the housing, which may serve as part of an implantable device, the cover preferably has a thickness of less than 1 mm, preferably less than 500 μm, more preferably of 400 μm or less.

On the other hand, in order to provide sufficient resistance during the hermeticity test, when the cover part is exposed to mechanical stress, e.g., due to application of a vacuum, the cover part needs to be solid enough to withstand that stress. Accordingly, the cover part may have a thickness of more than 200 μm, preferably of more than 300 μm. Most preferably, the cover part has a thickness of between 300-350 μm or between 370-430 μm. As one alternative for such a cover, a thin glass, e.g. comprising borosilicate glass, may be chosen. Alternative cover parts may comprise alternative materials without departing from the scope of the present invention, such as soda lime glass, quartz or vycor, among others.

In some embodiments, as for instance shown in FIG. 3, the cover part 60 on top of the base part 50 may comprise a beveled edge around a circumference of the cover part 60. The bevel angle preferably is within the range of about 60-80°, preferably 70-90°, particularly, the bevel angle is 70° and more particularly the bevel angle is 80°. Such a slanted edge may reduce the risk of damaging tissue or devices due to a sharp edge.

FIG. 4 shows an exploded view of the base part 50 of the hermetic housing 40. The bottom layers 56, 53, 54 provide a bottom seal of the hermetic housing 40. Each layer may comprise a ceramic material, such as a low temperature co-fired ceramic (LTCC). The bottom layers 56, 53, 54 comprise metallizations and vias 57. In addition, the inner bottom layer 54 comprises electrical connections or connection pads 55. Electrical components, which are to be positioned within the housing 40, may be connected with the outside of the housing 40 by contacting the contact pads 55 and contacting through the metallizations and vias. The metallizations may for instance comprise gold. The contacts 55 on the inner bottom layer 54 may for instance comprise AgPd. The ring-like wall layers with the top wall layer 52 are provided without metallization.

On top of the top wall layer, the connecting means 70 is provided as a ring like layer. The inner diameter of the connecting means layer corresponds to the inner diameter of the ring-like top wall layer. In particular embodiments, during production of the base part 50 of the hermetic housing 40, the connecting means 70 is a solder paste, which is printed on the top wall layer 52.

In the particular embodiment of FIG. 4, the base part comprises a total of ten LTCC layers. The bottom four layers, including the outermost bottom layer 56, intermediate layers 53, and the innermost bottom layer 54, comprise metallizations and vias for contacting electrical components and providing electrical connections to the outside of the housing 40. The remaining six layers comprising the top wall layer 52 are provided to form the wall, or rim, of the housing 40. It will be noted however, that the number of layers for the bottom part of the base part 50 as well as the number of layers for the wall part of the base part 50 may differ from the above example. In particular, the number of layers depends on factors such as thickness, hermeticity of the respective layer, metallizations and via-sizes, intended heremticity, and others. In order to provide a hermetic sealing of the bottom of the housing 40, i.e., the four bottom layers according to FIG. 4, a total thickness of 500 μm may be sufficient to hermetically seal the housing. Depending on the hermetic standards, which shall be applied, the thickness may also be below or above 500 μm, as well.

In order to provide a cavity of sufficient size, the wall layers may have a total thickness, i.e. a height of the cylindrical cavity, of about 1200 μm. Again, depending on the specific application, the total height of the wall layers, i.e. the cavity, may be below or above 1200 μm, as well.

The connecting means 70, here a glass solder paste, is printed on top of the top wall layer 52. That solder paste may be a lead-free solder, such as SnBi-solder or, in particular, be a solder paste available under the trading name “Glass Solder Ferro DL 11-205”. Alternative solder materials may be used without departing from the scope of the present invention. In particular, lead-free, and, generally, metal-free solder pastes may be preferable.

During production of the base of the hermetic housing, the individual layers may be laminated and fired, in order to provide a tight and hermetic bond. In order to hermetically seal the base part 50 with the cover part 60, the connecting means 70, i.e. a heat-curable solder paste, is provided between the cover part 60 and the base part 50. Prior to covering the base part 50 with the cover part 60, the desired electronic components are placed and connected within the hermetic housing 40. The cover part 60 is then placed on the base part 50 with the solder paste sandwiched there between. The solder paste is then heated by means of light, preferably laser light, directed on the interface between base part 50 and cover part 60. The solder paste is cured due to the application of energy by the laser and provides a hermetic seal between the base part 50 and the cover part 60. It is to be noted that the laser light has a wavelength in that frequency range, in which the cover part is transparent for the light and the solder paste is absorbent for the light.

In order to prevent the housing from heating up to an undesirable amount due to thermal conductivity from the interface between the cover part 60 and the base part 50 in response to laser application, the housing, i.e. the ceramic bottom layers of the base part, is placed on a cooling plate. The cooling plate typically is set at a temperature of about 100° C. in order to prevent heating of the entire device and particular the bottom part of the base part, where electronic devices may be connected, to temperatures induced by the light-application.

Further, in order to improve the sealing of base part 50 and cover part 60, a weight may be applied on the cover part, in order to increase the pressure of the cover part 60 on the base part 50 and the connecting means 70 placed there between. That may allow better distribution of the connecting means 70, i.e. the solder paste according to preferred embodiments of the present invention, and thus an increase in contact area between the solder and the cover part and/or the base part. Further, that may allow an increased hermetic sealing due to a better bonding between connecting means 70 and both, the cover part 60 and the base part 50.

FIG. 5 shows a top view of an assembled hermetic housing 40. From that top view, the ring-shaped top wall layer 52 is shown. At an inner diameter of the top wall layer 52, the connecting means 70, i.e. the solder paste, is provided as a ring structure, wherein the outer diameter of the connecting means 70 is smaller than the outer diameter of the top wall layer 52. The outer diameter of the connecting means 70 may also be smaller than the outer diameter of the cover part 60, which is not shown in FIG. 5. A cavity formed by the wall layers on the bottom of the cavity is confined by the inner bottom layer 54. Connecting pads 55 on the inner bottom layer 54 are provided, which allow connection with, e.g., a stimulation chip 29 or other electrical components.

In some embodiments, a transmitting and/or receiving unit, e.g., a coil, may also be provided in the cavity and on the inner bottom layer 54.

### LIST OF REFERENCE SIGNS

1 retinal implant
2 eyeball
3 visual interface
4 first transmission coil
5 second transmission coil
6 pocket computer
7 wire connection
8 signal generation unit
9 temple arm
10 video camera
11 cornea
12 eye lens
13 retina
14 sclera
15 choroid membrane
16 iris
17 ciliary muscle
18 intraocular part
19 extraocular part
20 wire connection
21 eyeglass frame
22 glasses
23 first transmission coil
24 second transmission coil
25 temples
26 receiver coil
27 infrared beam
28 receiver element
29 stimulation chip
30 array
40 hermetic housing
50 base part
52 top wall layer
53 intermediate layer
54 inner bottom layer
55 contacts/connection pads
56 outer bottom layer
57 metallizations and vias
60 cover part
70 connecting means

1. An hermetic housing that is configured to be implanted in a body of an animal or a human patient, the housing comprising:
a base part;
a cover part configured to cover the base part; and
a connecting means, provided at an interface between the base part and the cover part, wherein the base part comprises a first hermetic material and the cover part comprises a second hermetic material,
wherein the connecting means comprises a third hermetic material, adapted to hermetically seal an interior of the hermetic housing from an outside of the hermetic housing.

2. The hermetic housing according to claim 1, wherein the cover part comprises a material, which is transparent to at least a predetermined wavelength or wavelength range, and wherein the material is a metal-free material.

3. The hermetic housing according to claim 1, wherein the base part comprises a ceramic material.

4. The hermetic housing according to claim 1, wherein the connecting means comprises a metal-free solder paste.

5. The hermetic housing according to claim 4, wherein the cover part comprises a material that is transparent to a predetermined wavelength or wavelength range, wherein the solder paste is light absorbent for the predetermined wavelength or wavelength range, or wherein the solder paste is light-absorbent at least for one wavelength or a part of the wavelength range, for which the cover part of the hermetic housing is transparent.

6. An electronics package for an implant device, the electronics package comprising
at least one receiving unit;
an electrical circuit adapted to generate a stimulating signal; and
a first hermetic housing,
wherein the first hermetic housing comprises:
a base part adapted to receive the electrical circuit and/or the at least one receiving unit;
a cover part, adapted to cover the base part; and
a connecting means, provided between the base part and the cover part, adapted to connect the base part and the cover part and to hermetically seal an interior of the hermetic housing from an exterior of the hermetic housing.

7. The electronics package according to claim 6, wherein the electronics package comprises a second hermetic housing arranged at least partially around the first hermetic housing.

8. The electronics package according to claim 6, wherein at least one transmitting and/or receiving unit is provided outside the first hermetic housing and inside the second hermetic housing.

9. The electronics package according to claim 6, wherein the second hermetic housing comprises a biocompatible material that includes, silicone.

10. The electronics package according to claim 6, wherein the base part comprises a bottom part, the bottom part comprising a stack of layers, wherein at least one of the layers in the stack of layers comprises an integrated electrical circuit.

11. A prosthesis system comprising:
a first implantable device comprising an electronics package, the electronics package comprising:
at least one receiving unit
an electrical circuit adapted to generate a stimulating signal; and
a first hermetic housing,
wherein the first hermetic housing comprises:
a base part adapted to receive the electrical circuit and/or the at least one receiving unit
a cover part, adapted to cover the base part; and
a connecting means, provided between the base part and the cover part, adapted to connect the base part and the cover part and to hermetically seal an interior of the hermetic housing from an exterior of the hermetic housing;
a second implantable device that is adapted to be implanted into an organ of a patient, and that is connected with the electronics package.

12. The prosthesis system according to claim 11, wherein the second implantable device comprises at least one stimulating electrode configured to stimulate living tissue or cells.

13. The prosthesis system according to claim 11, wherein the second implantable device comprises a receiving coil, which is connected to a transmitting coil of the electronics package.

14. The prosthesis system according to claim 11, wherein the prosthesis system comprises an extracorporeal component that includes:
a first transmitting unit; and
a signal generation unit adapted for generating a signal and applying the signal to the first transmitting unit, wherein the first transmitting unit is adapted for transmitting the signal generated by the signal generation unit to the electronics package.

15. A method for providing an implantable electronics package suitable to be implanted in a body of an animal or a human patient comprising:
providing a base part of a hermetic housing adapted to receive an electrical circuit and/or at least one receiving unit;
providing an electrical circuit and/or at least one receiving unit on a bottom part of the base part;
providing, on a surface of the base part, a connecting means;
providing, on the connecting means, a cover part;
connecting the cover part with the base part and hermetically sealing a gap between the cover part and the base part by light induced heating of the connecting means, such that an interior of the hermetic housing is hermetically sealed from the an exterior of the hermetic housing.

16. The method according to claim 15, wherein the connecting means comprises a glass solder paste, and wherein connecting the cover part with the base part comprises laser-soldering of the solder paste.

17. The method according to claim 16, further comprising pre-curing the solder paste onto the base part of the hermetic housing.

18. The method according to claim 15, further comprising controlling a light of a laser to heat an interface area between the cover part and the base part covered with the connecting means, wherein a light intensity of the light is increased and decreased linearly at least around a desired target intensity at a predetermined target point or target area at the interface between the cover part and the base part.

19. The hermetic housing according to claim 3, wherein the ceramic material is a metal-free ceramic material or wherein the base part comprises a plurality of ceramic layers that include layers of low temperature, co-fired ceramic (LTCC).

20. The hermetic housing according to claim 2, wherein the metal-free material includes metal-free glass.
